(19) United States
(12) Patent Application Publication
(10) Pub. No.: US 2007/0243142 A1
(43) Pub. Date: Oct. 18, 2007

(54) Dental liquid desensitizing preparation with resistant flow

(76) Inventor: John J. Discko, Trumbull, CT (US)

Correspondence Address: PAUL A. FATTIBENE;FATTIBENE AND FATTIBENE, 2480 POST ROAD, SOUTHPORT, CT, 06890, US

(21) Appl. No.: 11/403,098

(22) Filed: Apr. 12, 2006

(51) Int. Cl. A61K 8/81

(52) U.S. Cl. 424/49

(57) ABSTRACT

A composition applied to exposed dentin of a tooth comprising hydroxyethylmethacrylate or HEMA, glutaldehyde, and a thickening agent in aqueous solution. The thickening agent or viscosity enhancing material increases the viscosity of the composition, preventing the composition from moving out of position or dripping on a vertical surface. The present invention helps the composition from contacting or irritating soft tissue in the mouth of a patient. The composition decreases tooth sensitivity and improves bonding.

### FIELD OF THE INVENTION

The present invention relates in general to a dental desensitizer used in the treatment of teeth, and more particularly to a dripless or flow resistant dentin dental desensitizer.

### BACKGROUND OF THE INVENTION

A human tooth is generally covered with enamel which is hard and relatively insensitive. Under the enamel is a softer, more sensitive dentin material. When dentin material is exposed, a tooth often becomes sensitive. Dentin is typically exposed when caries, decay, excessive wear, or abrasion penetrates the enamel. In repairing or reconstructing the tooth, the sensitive dentin is often exposed. There are many pre-treatments to dentin that are used to improve bonding as well as to desensitize the dentin so as to prevent sensitivity after repair or reconstruction of the tooth. Various materials have often been used on dentin as a primer or liner composition to pre-treat the tooth prior to filling with a composite resin used for filling a cavity and repair of the tooth. The pre-treatments often comprise an ester of acrylic or methacrylic acid and an aldehyde.

One such primer or liner composition is disclosed in U.S. Pat. No. 4,593,054 entitled “Adhesive Promoting Agent, Process For Its Preparation and Use Thereof On Collageneous Material” issuing to Asmussen et al on Jun. 3, 1986. Therein disclosed is a composition containing thirty-five percent hydroxyethyl methacrylate, hereinafter HEMA, and five percent glutaraldehyde, in an aqueous solution.

Another pre-treatment material applied to dentin is disclosed in U.S. Pat. No. 4,814,423 entitled “Dual Bonding Adhesive Composition” issuing to Huang et al on Mar. 21, 1989. Therein disclosed is an ethylenically unsaturated polymerizable phosphate and an aldehyde used to precondition and improve bonding to dentin. The glutaraldehyde improves bonding to collagen and the phosphate improves bonding to calcium.

Another pre-conditioner or primer applied to dentin using HEMA and glutaraldehyde is disclosed in U.S. Pat. No. 5,525,648 entitled “Method For Adhering To Hard Tissue” issuing to Aasen et al on Jun. 11, 1996. Therein disclosed is a composition particularly adapted to application in a high humidity environment.

A tooth sensitivity treating composition is disclosed in U.S. Pat. No. 5,693,315 entitled “Mammal Tooth Treating Composition” issuing to Bevilacqua on Dec. 2, 1997. Therein disclosed is a tooth sensitivity treating composition containing HEMA, glutaraldehyde, and sodium fluoride.

Yet another dental desensitizing composition is disclosed in U.S. Pat. No. 5,797,749 entitled “Dental Composition” issuing to Bertolotti et al on Aug. 25, 1998. Therein disclosed is an aqueous based dental composition used for desensitizing an exposed tooth surface containing HEMA and benzethonium chloride. Benzethonium chloride is used instead of glutaraldehyde, because glutaraldehyde may cause irritation.

Another dental composition for the treatment of dentin is disclosed in U.S. Pat. No. 6,592,372 entitled “Method of Etching and Priming A Tooth” issuing to Jia et al on Jul. 15, 2003. Therein disclosed is a composition utilizing HEMA, glutaraldehyde, and an olefinically unsaturated-SO₃ terminated monomer.

There are many compositions that have been used to treat dentin for a variety of different reasons, mostly to improve bonding of a composite filling material and to decrease sensitivity of the tooth once filled. Most of these dental conditioning materials include HEMA and glutaraldehyde. These materials may irritate and even cause chemical burns to soft tissue, such as gingiva or gums surrounding the tooth. Most of these aqueous based formulations have a very low viscosity and tend to run or drip on the vertical surfaces of the tooth. Often this causes the composition to come in direct contact with the soft tissue or gingiva, which may cause irritation. Therefore, there is a need for an improved formulation of a chemical composition containing HEMA and glutaraldehyde used for bonding to and desensitizing dentin for use in dental procedures on teeth. Additionally, there is an increasing need for desensitizing dentin due to tooth enamel damage caused by excess bleaching of the teeth, which may lead to tooth sensitivity. Teeth are often bleached in an effort to obtain whiter teeth and improved appearance. Also, as gum lines recede in elderly patients, portions of the root or dentin may be exposed near the gum line causing sensitivity. This area is especially difficult to treat due to the proximity to the soft gum tissue and the low viscosity of prior desensitizing agents.

### SUMMARY OF THE INVENTION

The present invention comprises a tooth desensitizing agent, preferably composition of an ester of methacrylic acid and an aldehyde having a viscosity enhancing material or thickening agent. Preferably, the ester of methacrylic acid is hydroxyethyl methacrylate or HEMA and the aldehyde is glutaraldehyde and the viscosity enhancing material or thickening agent comprises silica, pectin, Xanthan gum, carbopol, or other compatible or suitable material. The viscosity enhancing material or thickening agent prevents the composition, when applied to a tooth, from migrating or dripping away from the area to which it has been applied so as to avoid irritating or damaging soft tissue surrounding the tooth. The thickening agent or viscosity enhancing material preferably results in the material maintaining its position for-a minimum of forty-five seconds on a vertical surface at an ambient temperature typically found in the mouth.

Accordingly, it is an object of the present invention to prevent irritation or damage to soft tissue in the mouth or surrounding a tooth.

It is an advantage of the present invention that it is easy to use.

It is another advantage of the present invention that it will remain in a fixed position and will not drip or run onto soft tissue, such as gingiva, tongue, cheeks, and lips.

It is a feature of the present invention that a viscosity enhancing material or thickening agent is used.

It is a feature of the present invention that it may be applied with a hand held applicator.

It is another feature of the present invention that it may be applied with a syringe.

These and other objects, advantages, and features will become more readily apparent in view of the following more detailed description.

### BRIEF DESCRIPTION OF THE DRAWINGS

FIG. 1 schematically illustrates application of the present invention to a cavity in a tooth.

FIG. 2 schematically illustrates application of the present invention with a syringe to the partially exposed root portion of a tooth.

### DETAILED DESCRIPTION OF THE PREFERRED EMBODIMENTS

The present invention is a composition to be applied to the dentin of a tooth or other hard tissue. An ester of methacrylate acid and an aldehyde in an aqueous solution is combined with a viscosity enhancing material or thickening agent so as to form a viscous liquid or gel. The composition has a viscosity such that it will not run on a vertical surface for a minimum of forty five seconds at a temperature typically found in the mouth or ranging from approximately ninety to one hundred degrees Fahrenheit or approximately thirty-two and thirty eight degrees Celsius. Preferably, the ester of methacrylate acid is hydroxyethylmethacrylate acid or a HEMA and the aldehyde is glutaldehyde. The thickening agent preferably is any inert agent that is compatible with HEMA, glutaldehyde and water. Preferable thickening agents may be fumed silica, pectin, xanthan gum, carbopol and other well known thickening agents. Other thickening agents may be selected from a large number of ingredients. As an example, the following thickening or gelling agents may be used, agar made from seaweed, propylene glycol alginate, alginate, carrageenan, cellulose derivatives, exudate gums such as gum arabic, gum tragacanth, gum karaya, gellan gum, gelatin, microcrystalline cellulose, collegen, konjac gum, modified starches, seed gums, or any of a number of other known thickening or gelling agents. The term thickening agent is used to mean any material that increases viscosity, increases resistance to flow, or prevents a composition from running or dripping.

In a preferred embodiment, the pre-treating composition includes 30% to 40% by weight of HEMA, 1% to 10% by weight of glutaldehyde, with the balance being water and the thickening agent. A specific preferred embodiment comprises 35% by weight HEMA and 5% by weight glutaldehyde and an aqueous solution containing the thickening agent. Small quantities of other materials may be added such as an anti-microbial; for example, benzalkonium chloride or an antidegenerate, such as di-tert-butyl para cresol. Additionally, sodium fluoride may be added for its known beneficial effects for enhancing anti-microbial action and strengthening and reinforcing a bond.

A sufficient quantity of thickening agent is utilized so as to prevent the composition from dripping or moving out of position on a vertical surface generally for a period of time between thirty to sixty seconds, and preferably at least forty-five seconds, at a temperature typically found in the mouth, or approximately between ninety and one hundred degrees Fahrenheit or approximately thirty-two and thirty eight degrees Celsius.

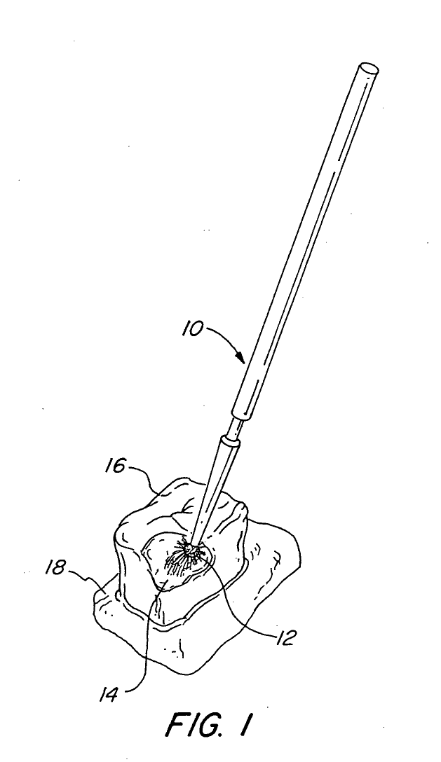

FIG. 1 schematically illustrates the application of the composition of the present invention to a tooth. An applicator 10 having an absorbent or liquid holding applicator end 12 may be used to contain and hold the composition of the present invention so as to apply it to a tooth 16 having an exposed dentin 14. The cavity formed in the tooth exposing the dentin 14 is generally filled with a composite resin so as to restore or reconstruct the tooth 16. Prior to filling with a composite resin, the applicator 10 is used to apply the composition of the present invention to the dentin 14. The viscosity of the composition of the present invention is adjusted with the aid of a thickening agent, such that the composition of the present invention will not move out of position from its application on the dentin 14 or vertical surface of the tooth 16 for a predetermined treatment time, preferably minimum of forty-five seconds. Generally, after this predetermined treatment time of generally not more than sixty seconds, but maybe up to several minutes, the composition may be rinsed from the tooth 16 and exposed dentin 14.

The preferred viscosity or thickness may also be determined with a viscometer. In testing it has been determined that a preferred viscosity ranges between 6600 to 9200 cP (centipoise) or Mpa·s (milliPascal-seconds). The testing was performed on a Brookfield viscometer model DV-I+. The Brookfield no. 7 probe was used at an RPM of ten at a temperature of approximately sixty-eight degrees Fahrenheit or twenty degrees Celsius.

Accordingly, the present invention prevents any irritation or damage to the soft tissue or gingiva 18, generally located around the tooth 16. Therefore, the present invention permits the use of beneficial materials such as HEMA and glutaldehyde that may be irritating or caustic to soft tissues, to be used and applied to the exposed dentin of a tooth without a likelihood of the composition migrating or dripping onto soft tissue. This is particularly advantageous when the composition of the present invention is applied in the very close and difficult to access areas of the mouth of a patient.

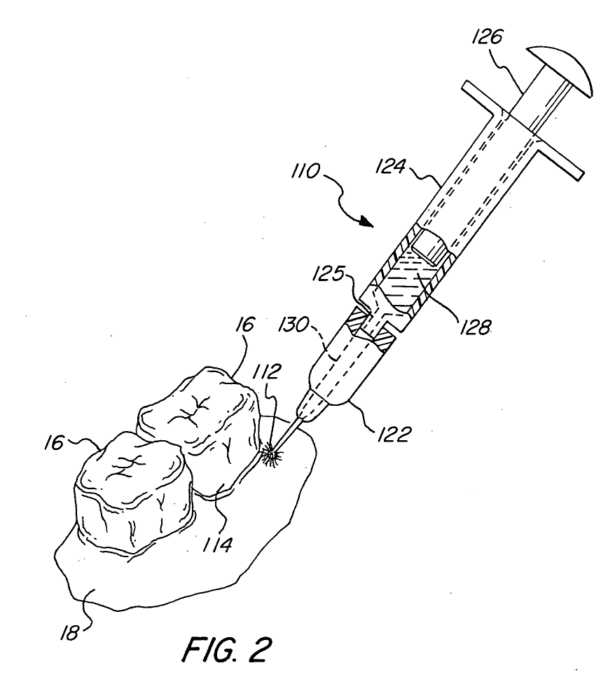

FIG. 2 illustrates the application of the material or composition of the present invention with a bulk syringe. The syringe applicator 110 has a cannula tip 122 connected to a syringe body 124. Placed within the syringe body is a desensitizing composition 128. A plunger 126 is used to extrude or force the desensitizing composition 128 through a passage 130 in the tip 122 and the applicator end 112 of the cannula tip 122. The applicator end may be made of any soft or absorbent material, such as flock, foam, or cotton. The desensitizing composition 122 must be sufficiently fluid so as to be forced through the applicator end 112. A preferred flowability or viscosity of the desensitizing composition 122 has been determined to be approximately 6600 to 9200 cP. This preferred viscosity provides sufficient flowability through the applicator end 112 and sufficient viscosity to stay in place on the tooth 16 for the desired time. This greatly facilitates application of the desensitizing composition and prevents or reduces the likelihood of the desensitizing composition coming into contact with the soft tissue in the mouth, such as the gingiva or gums 18.

In a typical application, illustrated in FIG. 2, the desensitizing composition 128 is applied with the bulk applicator 110 to exposed dentin at the root or gum line 114. Receding gums sometimes occur with disease or advanced age. The receding gums often expose a portion of the root of the tooth causing sensitivity. This sensitivity often causes pain or discomfort. A desensitizer is often used to aid in desensitizing the exposed root portion of the tooth, thereby providing reduced sensitivity to the patient.

While the present invention has been described with respect to various preferred embodiments, it will be appreciated to those skilled in the art that various modifications may be made without departing from the spirit and scope of the invention.

What is claimed is:

1. A tooth desensitizing composition comprising:
a tooth desensitizing agent; and
a thickening agent,
whereby the tooth desensitizing composition is prevented from migrating or running out of position once applied.

2. A tooth desensitizing composition as in claim 1 wherein said tooth desensitizing agent comprises:
an ester of metholacrylic acid; and
an aldehyde.

3. A tooth desensitizing composition as in claim 1 wherein:
said thickening agent is selected from the group consisting of silica, pectin, xanthan gum, and carbopol.

4. A tooth desensitizing composition as in claim 1 wherein:
said thickening agent is selected from the group consisting of agar made from seaweed, propylene glycol alginate, alginate, carrageenan, cellulose derivatives, exudate gums such as gum arabic, gum tragacanth, gum karaya, gellan gum, gelatin, microcrystalline cellulose, collegen, konjac gum, modified starches, and seed gums.

5. A tooth desensitizing composition as in claim 1 wherein:
said thickening agent is of a sufficient quantity so as to prevent movement of the composition on a vertical surface for at least forty-five seconds at a temperature between approximately ninety and one hundred degrees Fahrenheit or thirty-two and thirty eight degrees Celsius.

6. A tooth desensitizing composition as in claim 1 wherein:
said thickening agent is of a sufficient quantity so as to provide a viscosity of between 6600 and 9200 centipoise.

7. A tooth desensitizing composition as in claim 2 wherein said ester of metholacrylic acid comprises hydroxyethylmethacrylate and said aldehyde comprises glutaldehyde.

8. A tooth desensitizing composition as in claim 7 wherein said hydroxyethylmethacrylate comprises 30% to 40% by weight of the composition and said glutaldehyde comprises between 1% and 10% by weight of the composition.

9. A tooth desensitizing composition as in claim 8 wherein said hydroxyethylmethacrylate comprises substantially 35% by weight of the composition and said glutaldehyde comprises substantially 5% by weight of the composition.

10. A tooth desensitizing composition used in treating exposed dentin of a tooth comprising:
substantially 35% by weight of hydroxyethylmethacrylate;
substantially 5% by weight of glutaldehyde; and
a thickening agent of a sufficient quantity so as to prevent movement of the composition on a vertical surface for at least forty-five seconds at a temperature between approximately ninety and one hundred degrees Fahrenheit or thirty-two and thirty eight degrees Celsius.

11. A tooth desensitizing composition used in treating exposed dentin of a tooth as in claim 10 further comprising:
fluoride.

12. A tooth desensitizing composition used in treating exposed dentin of a tooth as in claim 10 wherein:
the tooth desensitizing composition is in an aqueous solution.

13. A tooth desensitizing composition used in treating exposed dentin of a tooth comprising:
35% by weight of hydroxyethylmethacrylate;
5% by weight of glutaldehyde;
a thickening agent selected from the group consisting of agar made from seaweed, propylene glycol alginate, alginate, carrageenan, cellulose derivatives, exudate gums such as gum arabic, gum tragacanth, gum karaya, gellan-gum, gelatin, microcrystalline cellulose, collegen, konjac gum, modified starches, seed gums, silica, pectin, xanthan gum, and carbopol; and
said thickening agent being of a sufficient quantity so as to prevent movement of the composition on a vertical surface for a minimum of forty-five seconds at a temperature between approximately ninety and one hundred degrees Fahrenheit or thirty-two and thirty eight degrees Celsius,
whereby the tooth desensitizing composition is prevented from migrating or running out of position once applied.

14. A tooth desensitizing composition and dispenser comprising:
a bulk syringe having a syringe body, a discharge end, and a plunger;
a tip having a porous applicator end attached to the discharge end; and
a tooth desensitizing composition having a viscosity between 6600 and 9200 centipoise, wherein the porous applicator end is adapted to be sufficiently porous so as to permit said tooth desensitizing composition to pass there though.

15. A tooth desensitizing composition and dispenser as in claim 14 wherein said tooth desensitizing composition comprises:
an ester of metholacrylic acid;
an aldehyde; and
a thickening agent.

16. A tooth desensitizing composition and dispenser as in claim 15 wherein:
said thickening agent selected from the group consisting of agar made from seaweed, propylene glycol alginate, alginate, carrageenan, cellulose derivatives, exudate gums such as gum arabic, gum tragacanth, gum karaya, gellan gum, gelatin, microcrystalline cellulose, collegen, konjac gum, modified starches, seed gums, silica, pectin, xanthan gum, and carbopol.

17. A method of dispensing and applying a tooth desensitizing composition to a tooth comprising:
placing a tooth desensitizing composition having a viscosity between 6600 and 9200 centipoise in an applicator having a porous applicator end;
forcing the tooth desensitizing composition out of the porous applicator end; and
applying the tooth desensitizing composition to a tooth with the porous applicator end,
whereby the tooth desensitizing composition is sufficiently fluid to pass through the porous applicator end and sufficiently viscous not to drip or run onto surrounding soft tissue.

18. A method of treating a tooth with a tooth desensitizing composition comprising the step of:
applying the tooth desensitizing composition having a viscosity between 6600 and 9200 centipoise to the tooth.

19. A method of treating a tooth with a tooth desensitizing composition as in claim 18 wherein:
said step of applying comprises applying the tooth desensitizing composition with an applicator.

20. A method of treating a tooth with a tooth desensitizing composition as in claim 18 wherein:
said step of applying comprises applying the tooth desensitizing composition with a syringe.

21. A method of treating a tooth with a tooth desensitizing composition as in claim 18 comprising the further step of:
removing the tooth desensitizing composition from the tooth within sixty seconds.
